(19) United States
(12) Patent Application Publication
(10) Pub. No.: US 2008/0228126 A1
(43) Pub. Date: Sep. 18, 2008

(54) Method of inhibiting disruption of the healing process in a physically modified stomach

(75) Inventor: Marc Bessler, Teaneck, NJ (US)

Correspondence Address: RODMAN RODMAN, 10 STEWART PLACE, SUITE 2CE, WHITE PLAINS, NY, 10603, US

(73) Assignee: THE TRUSTEES OF COLUMBIA UNIVERSITY IN THE CITY OF NEW YORK, New York, NY (US)

(21) Appl. No.: 11/458,721

(22) Filed: Jul. 20, 2006

Related U.S. Application Data
(60) Provisional application No. 60/785,104, filed on Mar. 23, 2006.

(51) Int. Cl. A61M 37/00; A61F 2/04

(52) U.S. Cl. 604/9; 623/23.7

(57) ABSTRACT

Methods for inhibiting disruption of the healing process in a hollow body organ such as a stomach that is physically modified to restrict or reduce its food receiving capability are described. A physical boundary is used to form the stomach modification. An inlet portion of the modified stomach communicates with the esophagus and an outlet portion of the modified stomach communicates with the small intestine. A bypass shunt is inserted into the modified stomach alongside the physical boundary. The shunt enables food to pass from the esophagus through the shunt to the small intestine thereby reducing food and fluid contact with the physical boundary, and reducing pressure and tension forces at the physical boundary that establishes the stomach pouch. The shunt is inserted during the stomach modification procedure.

### CROSS-REFERENCE TO RELATED APPLICATIONS

This application is based on provisional application 60/785,104 filed Mar. 23, 2006.

### FIELD OF THE INVENTION

This invention relates to physical procedures for treatment of severe obesity wherein the stomach is physically modified to reduce the amount of food that can pass through the stomach, and more specifically to a prophylactic method for inhibiting disruption and/or preventing breakdown of the healing process of a physically modified stomach.

### BACKGROUND OF THE INVENTION

Physical modification of the stomach to reduce the food receiving capacity of the stomach can provide a long lasting solution to the problem of enabling the severely obese to reduce their food consumption. In most instances the severely obese will experience significant weight loss within one to two years after their stomach has been physically modified.

In one known physical modification of the stomach, the top of the stomach is partitioned by providing a physical boundary to create a relatively small pouch, which functions as the new stomach where food is digested. The physical boundary is provided within the stomach, by sutures, stapling or plications, to establish the newly formed small stomach pouch. The portion of the stomach beyond the physical boundary of the small stomach pouch receives substantially no ingested food from the esophagus.

The term “sutures” as used herein is intended to include stapling, plications or other procedures as a means for forming a physical boundary.

The physically created relatively small stomach pouch (hereinafter referred to as the “stomach pouch”) bypasses the majority of the stomach and, depending upon the type of stomach modification, can also bypass a portion of the small intestine.

The suture path is one form of physical boundary that can be used to establish the stomach pouch. The suture path can extend from a proximal end of the stomach, such as the fundus, and terminate before a distal end of the stomach, such as the antrum. Termination of the suture path before the distal end of the stomach enables the stomach pouch to maintain some communication with the bypassed portion of the stomach.

The physically created stomach pouch aligns with and communicates with the esophagus and also communicates with the small intestine. The stomach pouch essentially reduces the food receiving capacity of a normal stomach to the volume of the stomach pouch created by the physical modification of the stomach.

It has been found that because the stomach pouch has a smaller capacity for receiving food than that of the normal stomach, a patient undergoing physical stomach modification will experience a sensation of satiation after consuming a relatively smaller quantity of food than was usually eaten before the physical modification. Once a patient achieves a sense of fullness with a lesser amount of food than was customary prior to the physical modification of the stomach, the patient will generally reduce food consumption during meal time and experience a gradual loss of weight.

After the stomach has been physically modified, the healing process at the physical boundary can take from about several weeks to about several months. During the healing process the suture path is usually exposed to food and digestive juices including gastric acid. The suture path is also subjected to the normal pressures and tensions that build up within the stomach pouch as a characteristic of the digestion process and peristalsis.

Digestive activity in the stomach pouch generally results in contact between the digestive juices and the suture path that establish the stomach pouch. Also, pressure build-up in the stomach pouch attributable to the digestive process and peristalsis can result in pressure and tension forces on the stomach tissue at the suture path. Thus, the exposure of the suture path to digestive activity in the stomach pouch and to contraction and relaxation of the musculature of the stomach caused by internal pressures, tension forces and peristalsis can disrupt the healing process of the stomach tissue in the region of the suture path.

It is thus desirable to inhibit disruption of the healing process and thereby facilitate the healing process.

### SUMMARY OF THE INVENTION

The present invention includes a method of inhibiting and/or preventing disruption or breakdown of the healing process in a physical modification of a hollow organ such as the stomach.

In one aspect of the invention a section of the stomach is bypassed by a relatively small stomach pouch. The stomach pouch includes inlet and outlet portions, with the inlet portion in communication with the esophagus and the outlet portion closer to the small intestine than the inlet portion. The physical modification of the stomach also substantially reduces communication between the esophagus and the portion of the stomach that is bypassed.

Healing is facilitated at the physical boundary that establishes the stomach pouch by inserting a bypass shunt of generally closed cross-sectional periphery into the stomach pouch alongside the physical boundary, such as alongside the suture path.

The shunt is inserted during the procedure for modifying the stomach to avoid subjecting a patient to a separate shunt insertion procedure. If the shunt is not inserted when the physical modification of the stomach is performed, healing at the suture path may be disrupted by the normal digestive process within the modified stomach. Potential disruption of the healing process can be avoided by timely insertion of the shunt during the physical modification of the stomach.

The shunt is of a length that extends from a point at or above the inlet portion of the stomach pouch or beyond the esophagus, to a point that is at or beyond the outlet or distal portion of the stomach pouch. The shunt thus enables food to pass through the shunt from the esophagus to the small intestine with relatively little or no food contact with the stomach sutures that establish the stomach pouch.

The healing portion of the stomach at the suture path is protected by the shunt from the digestive activity in the stomach pouch including pressure and tension forces that are characteristic of normal digestive activity.

In accordance with the invention the physical boundary in the stomach that establishes the stomach pouch, such as the suture path, is immediately shielded and substantially isolated from the digestive activity and pressure buildup that occurs in the stomach pouch when food is ingested. The inventive method thus helps to inhibit disruption and prevent breakdown of the healing process at the beginning of and throughout the healing process, and also facilitates the healing of the physical boundary that establishes the stomach pouch.

### DESCRIPTION OF THE DRAWINGS

In the accompanying drawings,

FIG. 1 is a simplified partial cross-sectional view of a stomach that has been physically modified to form a stomach pouch;

FIG. 2 is a simplified partial cross-sectional view of a bypass shunt installed in a stomach pouch in accordance with the inventive method;

FIG. 3 is a simplified sectional view taken on the line 3-3 of FIG. 2;

FIG. 4 is a simplified partial cross-sectional view of one embodiment of a bypass shunt device used in carrying out the method of the invention;

FIG. 5 is a simplified partial cross-sectional view showing a delivery catheter used for installation of the bypass shunt in the stomach; and,

FIG. 6 is a simplified partial cross-sectional view of another embodiment of a bypass shunt used in carrying out the method of the invention.

Corresponding reference numbers indicate corresponding parts throughout the several views of the drawings.

### DETAILED DESCRIPTION OF THE INVENTION

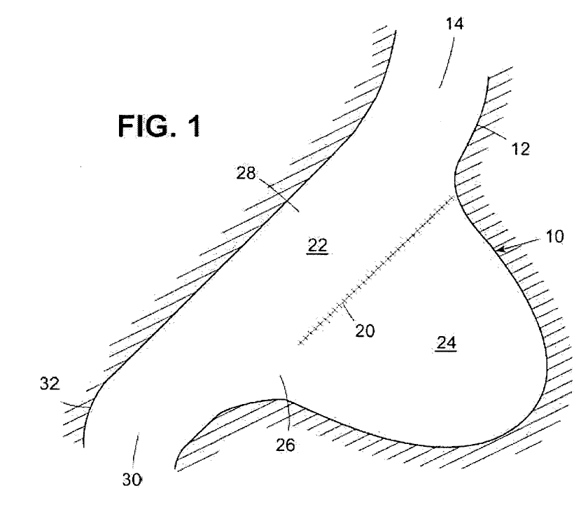

Referring to the drawings, FIG. 1 is a simplified sectional view of a stomach 10 that has been physically modified, such as by using gastric bypass surgery or plications.

Before physical modification of the stomach 10, food normally enters the stomach from the esophagus 12 (FIG. 1) through an inlet opening 14 at the approximate junction of the gastric and esophageal mucosa (gastro-esophageal junction) where the esophagus 12 joins the stomach 10.

The stomach 10, after physical modification, is provided with a physical boundary, such as a suture path 20 (FIG. 1), that divides the stomach 10 into a stomach pouch 22 of smaller size than the original stomach, and a stomach portion 24 that is substantially bypassed by the stomach pouch 22. The stomach pouch 22 communicates with the esophagus 12 in the same manner as the stomach 10 before the physical modification. However the stomach portion 24 has substantially reduced communication with the esophagus 12 at a section of the stomach 10 below the suture path 20 indicated by the reference number 26 (FIG. 1).

Thus, the inlet opening 14 at the gastro-esophageal junction of the original stomach 10 is maintained as the inlet opening to the stomach pouch 22. Food entering the inlet opening 14 substantially bypasses the stomach portion 24.

Food passes through the interior space 28 of the stomach pouch 22 (FIG. 1) and exits through an outlet opening 30 of the stomach pouch 22, which can be approximately at the pyloric opening to the duodenum 32.

The physical modification of the stomach 10 to form a stomach pouch having reduced food receiving capacity can be carried out using modification methods that are within the skill of the art of medical professionals who practice obesity surgery. The present invention is thus applicable to procedures for modifying a stomach to restrict the passage of food into the stomach or reduce the food receiving capacity of the stomach. For example, although not shown, the esophagus at the pyloric opening can be restricted in a known manner by physical modification using plications.

It should also be noted that in some of the known procedures for physical modification of the stomach 10, some portion of the stomach that is bypassed can be removed (not shown).

During digestion, food that is impelled by the esophagus 12 into the stomach pouch 22 (FIG. 1) is mixed with gastric juices in the stomach pouch and converted into chyme.

Gastric juice, which is of an acidic nature, and chyme, and any other stomach secretions or bodily-formed fluids in the stomach pouch 22 are collectively referred to as the stomach contents. During digestion of food in the stomach pouch 22 the stomach contents can come into contact with the suture path 20 that forms the physical boundary that establishes the stomach pouch 22. Such contact can cause irritation of stomach tissue at the suture path 20 and retard the formation of scar tissue at this surgical boundary during the post-modification healing period.

The digestive processes that take place in the stomach pouch 22, also result in a normal build up of pressure that can cause the stomach pouch 22 to periodically peristalse, contract and relax thereby producing intermittent tension and pressure at the suture path 20.

Contact of the stomach contents with the suture path 20 during the post modification healing period can thus cause discomfort to the patient and disrupt the healing process at the suture path 20.

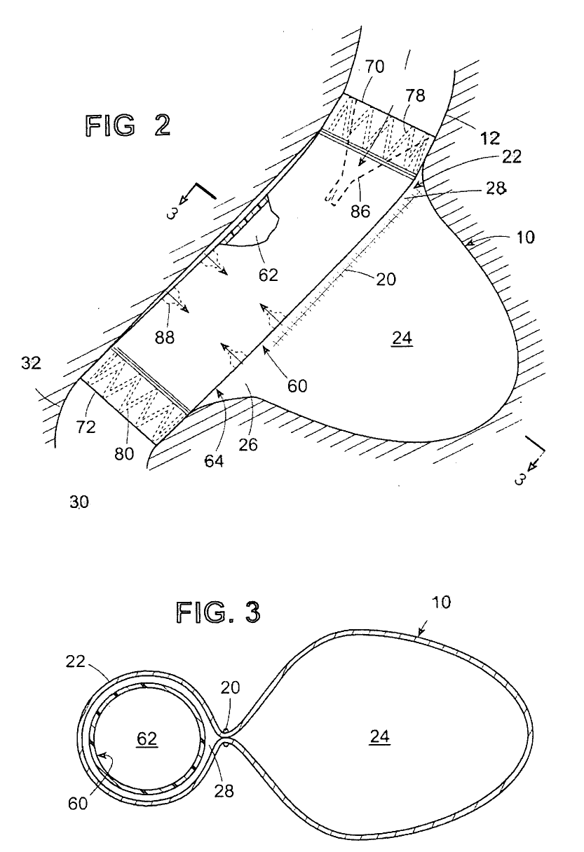

I believe that some of the discomfort a patient experiences after stomach modification can be reduced and that disruption of the healing process can be inhibited by installing a shunt device such as the shunt 60 (FIG. 2) in the stomach pouch 22 during the stomach modification. The shunt device 60 is provided with an inlet portion 70 to receive substantially all of the food that would normally enter the stomach pouch 22 following stomach modification.

The shunt device 60 has an interior space 62 (FIGS. 2, 3 and 4) through which ingested food is directed. The shunt device 60 is thus a conduit and also has an outlet portion 72 that communicates with the small intestine at a selected location to enable the shunt device 60 to pass ingested food into the small intestine, such as, for example, the pyloric opening to the duodenum 32 (FIG. 2).

By inserting the shunt device 60 in the stomach pouch 22, I can inhibit or prevent ingested food from contacting the suture path 20. I can thus use the shunt device 60 to shield the suture path 20 from a substantial amount of digestive activity to inhibit disruption of the healing process and thereby facilitate the healing process at the suture path 20. Thus, at least a major portion of the food that would normally enter the stomach pouch 22 and contact the suture path 20 is enabled to bypass the suture path 20.

The shunt device 60 thus facilitates the healing process at the suture path 20 because of substantially reduced exposure of the suture path 20 to the stomach contents that could otherwise cause irritation of stomach tissue at the suture path 20. The shunt device 60 also substantially confines to the interior space 62 (FIGS. 2, 3 and 4) of the shunt device 60 the digestive activity that would ordinarily take place within the stomach pouch 22. As a result there is reduced pressure buildup outside the shunt device 60 at the suture path 20.

Since the peristaltic activity at the stomach pouch 22 that is characteristic of digestion in the stomach pouch 22 is reduced by the shunt 60, tension and pressure forces at the suture path 20 are correspondingly reduced by the presence of the shunt 60 in the stomach pouch 22. Consequently, the discomfort that a patient might experience during the healing process because of peristaltic activity and irritation of the stomach tissue at the suture path 20 should be significantly alleviated by the shunt 60. Decreased pressure in the stomach pouch 22 also prevents poor healing at the suture path 20.

The shunt device 60 can be a gastric bypass device in the form of a self-expanding or other type of stent or fixation device that is inserted into the stomach in a collapsed condition and is expanded in any suitable known manner. The shunt device 60 can also cooperate with suitable known expansion devices such as a delivery catheter which may be used to help promote expansion of the stent.

The shunt device 60 is made of any suitable known biocompatible material, such as but not limited to stainless steel or nitinol. Alternatively the shunt device can be made of a suitable biodegradeable material such as, but not limited to thermoplastic oxyalkanoyl polymers, for example, epsilon caprolactone polymers, copolymers of L(−)lactide and glycolide, or terpolymer combinations thereof as disclosed in U.S. Pat. No. 5,085,629 to Goldberg et al, which is incorporated by reference herein.

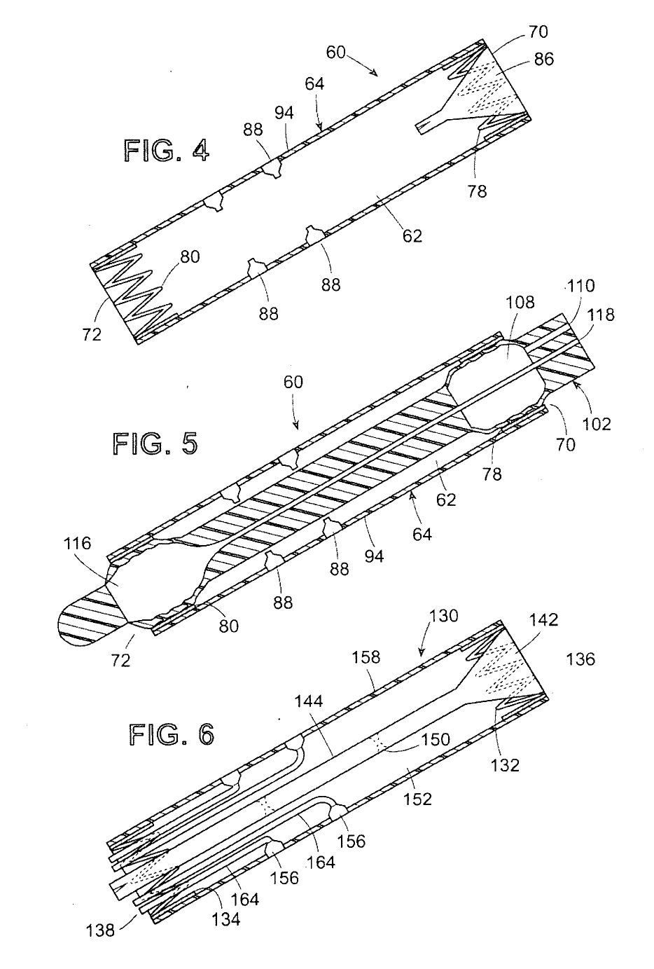

Referring to FIG. 4, the shunt 60 has the general shape of a tubular conduit 64 with an open inlet portion 70 at one end and an open outlet portion 72 at an opposite end. The conduit 64 has a generally closed cross-sectional periphery, and can have a cross-sectional shape other than circular, such as an oval or another selected configuration based on the organ at issue

An expandable stent member 78 (FIGS. 2 and 4) is provided at the inlet portion 70 and an expandable stent member 80 is provided at the outlet portion 72 of the conduit 64. The stent members 78 and 80 enable the shunt 60 to respectively expand against the inner surface of the esophageal junction 14 and the pyloric opening 30 to the duodenum 32 for stable positioning therein. If desired, suturing or other suitable known fixation, sealing or retention means can be provided to reinforce the fixed positioning of the proximal and distal end portions at the inlet and outlet portions 70 and 72 of the shunt 60.

A one way valve 86 (FIG. 4) of any suitable known construction, such as a known sock-type or a known duck-bill valve, can be provided at the inlet portion 70 to prevent acid reflux and also to prevent any other stomach contents from exuding back into the esophagus 12 from the shunt 60. Additional one way valves 88 (FIG. 4) that are smaller than the one way valve 86 but structurally and functionally similar to the known one way valve 86, can be provided in the stent wall 94.

The valves 88 are preferably closer to the outlet portion 72 than the inlet portion 70. The one way valves 88 permit entry of gastric juices from the stomach pouch 22 and also permit emptying of secretions from the stomach pouch 22 into the space 62 of the shunt 60, to allow the digestive process to begin within the shunt 60. The one way valves 88 also relieve pressure buildup outside the shunt 60 by venting the pressure into the shunt 60 to thereby transfer such pressure away from the stomach pouch 22.

The length and the diameter of the invention such as the shunt 60, depends upon the physical characteristics of the patient in whom the shunt is located. For example, the shunt 60 can generally be long enough to extend from the gastro-esophageal junction of a patient to the pyloric opening of the duodenum. Radial expansion requirements of the shunt 60 at the stent members 78 and 80 are usually based on anatomical data measurements well known to those skilled in the art.

Although the shunt 60 can have the shape of a tubular conduit, other cross-sectional configurations of regular or irregular shape can also be used, such as oval or another selected configuration based on the organ at issue. The shunt 60 can also have a lengthwise curvature.

With regard to dimensional parameters, the overall length of the shunt 60 for the stomach pouch 22 from the inlet end 70 to the outlet end 72 is at least equal to, and preferably greater than the length of the suture path 20. For example, the overall length of the shunt 60 can vary from about 1 or 2 centimeters to a dimension corresponding to the entire length of the intestinal tract and generally from about 5 to about 30 centimeters. The expanded diameter of the stent 60 can vary from less than 1 cm to about 20 centimeters, and is preferably from about 2 to about 3 centimeters. The sizes selected should enable the shunt 60 to functionally remain in a fixed position with or without suturing or other known methods of stabilizing the desired position of the shunt 60 in the stomach pouch 20.

The number, size and distribution of one way valves 88 (FIG. 4) provided in the conduit wall 94 permit emptying of fluids and mucous produced in the organ, without allowing significant pressure buildup. The central anti-reflux valve 86 is likely to be a single valve. The number of valves 88 also depends on the overall length of the shunt 60 and the anticipated volume and flow rate of ingested food that is directed through the shunt 60.

Referring to FIGS. 2 and 4, the shunt 60 is located in the stomach pouch 22 in a suitable known manner, for example by using a suitable known endoscope (not shown), and can be constructed to self expand in a known manner. The shunt 60 can also be expanded with a delivery catheter. For example, a delivery catheter 102 (FIG. 5) of any suitable known construction can be located in the interior space 62 of the shunt 60, before the shunt 60 is endoscopically positioned in the stomach pouch 22. If a self expanding stent is used (not shown) a suitable known delivery system or controlled release mechanism would accompany installation of the stent.

The delivery catheter 102 (FIG. 5) includes a dilation balloon 108 aligned with the inlet end portion 70 of the shunt 60 at the stent member 78. The dilation balloon 108 communicates with an inflation lumen 110 that directs fluid into the dilation balloon 108 to expand the balloon 108 and the stent member 78 to thereby hold the inlet end 70 of the shunt 60 at the gastro-esophogeal junction 14 (FIG. 2) or any other suitable location leading into the stomach pouch 22 from the esophagus 12.

The delivery catheter 102 (FIG. 5) further includes a dilation balloon 116 located at the outlet end portion 72 of the shunt 60 in alignment with the stent member 80. The dilation balloon 116 communicates with an inflation lumen 118 to expand the stent member 80 at the outlet end portion 72 and thereby hold the outlet end portion 72 at a desired position in or proximate to the stomach pouch 22.

The outlet end portion 72 of the shunt 60 can thus be held by the stent member 80 at the pyloric opening 30 (FIG. 2) of the stomach pouch 22. If desired, the inlet and outlet end portions 70 and 72 of the shunt 60 can also be held in place with any suitable known retention means such as sutures, stapling, hooks or other equivalent rentention methods known in the art.

After the dilation balloons 108 and 116 of the delivery catheter 102 (FIG. 5) have expanded the stent members 78 and 80 into a stable holding position the dilation balloons 108 and 116 are deflated and the delivery catheter 102 is removed from the shunt 60 in any suitable known manner. The one way valve 86 (FIG. 4) can be located at the inlet end 70 of the shunt 60 when the shunt is installed. The one way valve 86 serves to inhibit or prevent stomach acid reflux.

When there has been a satisfactory healing of the stomach tissue at the suture path 20, as determined by a medical professional, the shunt device 60 can be removed using suitable procedures, such as but not limited to endoscopy or surgery.

As an alternative to physical removal of the shunt 60, and as previously described, the shunt 60 can be constructed of a biodegradable material in a manner that will be apparent to those skilled in the art to break down and disintegrate over a selected period of time for harmless excretion or harmless absorption in the body tissue.

The shunt 60 can thus be structured to have an approximate viable life of predetermined time duration such as four to twelve weeks or more that corresponds to the expected healing period at the suture path 20 before breaking down.

Another embodiment of a shunt device for use in carrying out the method of the invention, is generally indicated by the reference number 130 in FIG. 6. The shunt device 130 includes stent members 132 and 134 at respective inlet and outlet portions 136 and 138. The stent members 132 and 134 are structurally and functionally equivalent to the stent members 78 and 80. A one-way valve 142 is provided at the inlet portion 136. The one-way valve 142 is generally functionally and structurally analogous to the one-way valve 86 (FIG. 4), but includes an elongated valve portion 144 that extends up to or beyond the outlet end portion 138 of the device 130.

The one-way valve 142 can also include openings 150 to permit a controlled egress of ingested food into the interior space 152 of the device 150 from the valve 142. The shunt device 130 further includes one-way valves 156 in the conduit wall 158. The one-way valves 156 are functionally analogous to the one-way valves 88 (FIG. 4) of the device 60 but have an elongated valve portion 164 that can extend up to or beyond the outlet end portion 138 of the shunt 130.

Under this arrangement, food passing through the shunt 130 is subjected to a reduced amount of digestive activity in comparison with that of the shunt 60 since the valves 156 will admit gastric juices that do not contact the food passing through the valve 142 until the food and gastric juices reach the outlet end portion 138 of the shunt device 130 and enter the small intestine

This invention is also adaptable to physical modification of other hollow organs of the body, wherein sutures, stapling, plications, and/or other procedures are used for physical modification of such organs. Non-limiting examples of hollow organs, the modification of which is adaptable to this invention include, the esophagus, the small intestine, the colon, the bile duct and the pancreas duct. For these other aspects of the invention dimensions of the shunt are based on the particular hollow organ in which the shunt is disposed and the extent of the physical modification of the organ.

As various changes can be made in the above constructions and methods without departing from the scope of the invention, it is intended that all matter contained in the above description or shown in the accompanying drawings shall be interpreted as illustrative and not in a limiting sense.

1. A method of inhibiting disruption or breakdown of the healing process in a physical modification of a stomach, comprising
(a) physically modifying the stomach to restrict or reduce the food receiving capacity of the stomach and wherein the modified stomach has inlet and outlet portions, and the inlet portion communicates with the esophagus, and the outlet portion communicates with the small intestine such that the esophagus communicates with the small intestine through the modified stomach, and
(b) inserting a shunt of generally closed cross-sectional periphery into the stomach during the stomach modification such that the shunt extends to the inlet and outlet portions of the modified stomach to enable food to pass through the shunt from the esophagus to the small intestine to reduce food and fluid contact with any physical boundary that establishes the stomach pouch and reduce any bodily pressure and tension forces due to the digestive process at any physical boundary that establishes the stomach pouch.

2. The method of claim 1 including modifying the stomach by forming a stomach pouch of smaller volume than the normal stomach.

3. The method of claim 2 wherein physical modification of the stomach includes using a stent member at the inlet portion of the stomach pouch to hold the shunt in position at the inlet portion.

4. The method of claim 1 wherein physical modification of the stomach includes using an endoscope to position the shunt at the inlet and outlet portions of the modified stomach.

5. The method of claim 1 wherein the shunt includes a one way valve at an end of the shunt where the shunt communicates with the esophagus.

6. The method of claim 1 wherein the shunt includes at least one one-way valve in a surface portion of the shunt to permit flow into the conduit through the surface portion of the shunt.

7. The method of claim 1 wherein the shunt is formed of a material that breaks down in the modified stomach after a predetermined time to permit elimination of the broken down shunt material as bodily waste.

8. A method of inhibiting disruption or breakdown of the healing process in a physical modification of a stomach of a patient, comprising,
(a) physically modifying a stomach by providing a physical boundary to form a stomach pouch of relatively smaller volume than the volume of the stomach before the physical modification, and wherein the stomach pouch has inlet and outlet portions, and wherein the inlet portion communicates with the esophagus, and the outlet portion communicates with the small intestine, and
(b) inserting a shunt of generally closed cross-sectional periphery into the stomach pouch alongside the physical boundary that establishes the stomach pouch, wherein the shunt extends from the esophagus to at least a selected portion of the small intestine to provide a food passage conduit that enables food to pass from the esophagus through the shunt to the small intestine to reduce food and fluid contact with the physical boundary that establishes the stomach pouch and reduce any bodily pressure and tension forces due to the digestive process at the physical boundary that establishes the stomach pouch.

9. The method of claim 8 wherein physical modification of the stomach includes establishing the physical boundary by using at least one of the following selected from the group consisting of sutures, staples, and plication.

10. The method of claim 8 wherein physical modification of the stomach includes using a stent member to hold the shunt in position at the inlet portion of the stomach pouch.

11. The method of claim 8 wherein physical modification of the stomach includes using an endoscope to position the shunt at the inlet and outlet portions of the stomach pouch.

12. The method of claim 8 wherein the shunt includes a one way valve at an end of the shunt where the shunt communicates with the esophagus.

13. The method of claim 8 wherein physical modification of the stomach includes installing the shunt during the stomach modification procedure.

14. The method of claim 8 wherein the shunt is formed from a biodegradable material that breaks down in the stomach pouch to permit elimination from the patient as waste material.

15. The method of claim 8, wherein the biodegradable material is at least one selected from the group consisting of thermoplastic oxyalkanoyl polymers, lactide polymers, and glycolide polymers.

16. The method of claim 15, wherein the biodegradable material breaks down after a predetermined period of time corresponding to the time needed to permit healing of the physical boundary.

17. A method of inhibiting disruption or preventing breakdown of the healing of a stomach that has been physically modified in a patient to reduce the food receiving capacity of the stomach, comprising,
(a) physically modifying a stomach to form a stomach pouch of relatively smaller volume than the volume of the stomach before physical modification, and wherein the stomach pouch has inlet and outlet portions, and wherein the inlet portion of the stomach pouch communicates with the esophagus and the outlet portion of the stomach pouch communicates with the small intestine, and
(b) inserting a conduit having an entry and an exit, into the stomach pouch, wherein the entry communicates with the esophagus and the exit communicates with the small intestine, wherein said conduit provides a passageway for food to pass directly from the esophagus to the small intestine to reduce food and fluid contact at any physical boundary that establishes the stomach pouch and reduce any bodily pressure and tension forces due to the digestive process at the physical boundary that establishes the stomach pouch.

18. The method of claim 17, wherein the conduit is a stent.

19. The method of claim 17, including using an endoscope to position the conduit in the stomach pouch.

20. A method of inhibiting disruption or preventing breakdown of the healing of a hollow organ of the body that has been physically modified in a patient comprising,
(a) physically modifying the hollow organ of the body at a predetermined length or predetermined area of the organ by changing the size, shape, or structure of the hollow organ at the predetermined length or predetermined area of the hollow organ such that the modified hollow organ has a proximal open portion leading into the predetermined length or predetermined area of the modification, and a distal open portion directed away from the predetermined length or predetermined area of the modification, and
(b) inserting a conduit, having a fluid entry portion and a fluid exit portion and a length that at least corresponds to the distance between the proximal open portion and the distal open portion of the modified hollow organ, into the modified hollow organ, such that the fluid entry portion and the fluid exit portion respectively communicate with body portions proximal to and distal to the proximal open portion and the distal open portion of the modified hollow organ, to enable the conduit to provide a passageway for bodily fluid to pass from the proximal open portion of the modified hollow organ to at least the distal open portion of the modified hollow organ to reduce bodily fluid contact at any physical boundary that establishes the modified hollow organ of the body and reduce any bodily pressure and tension forces at any physical boundary that establishes the modified hollow organ of the body.
